(19) United States
(12) Patent Application Publication
(10) Pub. No.: US 2026/0283472 A1
(43) Pub. Date: Sep. 24, 2026

(54) SYSTEMS AND METHODS TO MONITOR NOCICEPTION, PAIN, AND PAIN RELIEF

(71) Applicants: The General Hospital Corporation, Boston, MA (US); Massachusetts Institute of Technology, Cambridge, MA (US)

(72) Inventors: David Borsook, Northampton, MA (US); Ke Peng, Boston, MA (US); Keerthana Deepti Karunakaran, Boston, MA (US); Stephen Green, Cambridge, MA (US)

(21) Appl. No.: 19/477,039

(22) PCT Filed: Apr. 18, 2024
(86) PCT No.: PCT/US2024/025117

§ 371 Date: Oct. 20, 2025

Related U.S. Application Data
(60) Provisional application No. 63/496,733, filed on Apr. 18, 2023.

(51) Int. Cl. A61B 5/00

(52) U.S. Cl. CPC A61B 5/0075; A61B 5/4824; A61B 5/6803; A61B 5/7267; A61B 5/7282

(57) ABSTRACT

Systems and methods are provided for monitoring nociception and/or pain in a patient. The system includes optodes configured be placed on the patient's scalp and forehead to acquire functional near-infrared spectroscopy (fNIRS) data from the medial frontopolar cortex (mFPC) region and the somatosensory (SI) regions of the patient. The system also includes a control unit coupled to the optodes. The control unit is to obtain the acquired fNIRS data from the optodes, process the acquired fNIRS data, and recognize signatures in the fNIRS data associated with a pain event in real time, where the signatures include measures of dissociation between the fNIRS data from the mF-PC region and the fNIRS data from the SI region. The control unit is also configured to output a pain event classification to a user based on recognizing the signatures.

### CROSS-REFERENCE TO RELATED APPLICATIONS

This application is based on, claims priority to, and incorporates herein by reference for all purposes, U.S. Provisional Application Ser. No. 63/496,733, filed Apr. 18, 2023.

### BACKGROUND

An estimated 310 million surgical operations are performed worldwide annually. Surgery, including routine procedures, “attacks” the nervous system through peripheral nerve damage and consequent inflammatory processes. Acute pain following surgery can occur immediately after surgery to up to three months post operation. Progression to moderate or severe chronic postsurgical pain (i.e., greater than three months after surgery) occurs in 10-50% of patients. A number of clinical predictors likely contribute to pain chronification, such as intraoperative pain load, pre- and post-operative pain intensity, hyperalgesia/allodynia, and peri-surgical psychological factors such as pain catastrophizing. These reflect a combination of processes that can result in central sensitization.

At present, a serious gap in postsurgical pain prevention is attributed to the lack of pain/nociception treatment provided during the perioperative period (i.e., pre-, intra-, and post-surgery). During surgery, general anesthesia provides a state of unconsciousness but not adequate ongoing analgesia; the latter is usually supplemented on a clinical basis without a validated objective marker that anesthesiologists can monitor. Accordingly, a patient that is unconscious cannot report pain, yet the inability to communicate does not negate the pain experience. The inability to effectively communicate pain, and pain relief, is also an issue outside of surgery, such as for individuals like neonates/newborn, stroke patients, Alzheimer's patients, etc. as well as for chronic pain patients for determining more objective measures of pain or analgesia.

With respect to surgery monitoring, commercial EEG-based anesthesia monitors have been on the marketplace for several decades. However, existing devices have focused on monitoring a patient's level of consciousness and not nociception (i.e., experiencing and physiologically responding to noxious stimuli). That is, nociceptive afferent signals, such as those activated during surgery, have been shown to reach the brain under general anesthesia. Yet monitoring nociception is something that existing technologies are unable to do.

Thus, there is a need for systems for monitoring nociception that can provide an objective indication of nociception/pain during surgery. There is a further need for such monitoring pain and treatment efficacy in painful procedures, psychological, pharmacological, or instrumental treatments.

### SUMMARY

In accordance with one aspect of the disclosure, a system is provided for monitoring nociception and pain in a patient. The system includes optodes configured be placed on the patient's scalp and forehead to acquire functional near-infrared spectroscopy (fNIRS) data from the medial frontopolar cortex (mFPC) region and the somatosensory (S1) regions of the patient. The system also includes a control unit coupled to the optodes. The control unit is to obtain the acquired fNIRS data from the optodes, process the acquired fNIRS data, and recognize signatures in the fNIRS data associated with a pain event in real time. The signatures can include measures of dissociation between the fNIRS data from the mFPC region and the fNIRS data from the S1 region. The control unit is also configured to output a pain event classification to a user based on recognizing the signatures.

In accordance with one aspect of the disclosure, a system is provided for monitoring nociception and/or pain in a patient during surgery. The system includes a control unit, a first input device, a second input device, a third input device, and an output device. The first input device is to provide event detection information corresponding to a surgical event to the control unit. The second input device is to provide physiological data to the control unit. The third input device is to provide functional near-infrared spectroscopy (fNIRS) data from the medial frontopolar cortex region and the somatosensory regions of the patient to the control unit. The output device is to provide information to a user. The control unit is to process the event detection information, the physiological data, and the fNIRS data to obtain processed data, input the processed data to a machine learning classifier that classifies the surgical event as a pain event or a no-pain event, and control the output device to provide the classification from the classifier to the user.

In accordance with one aspect of the disclosure, a method is provided for detecting nociception and pain in a patient. The method includes obtaining functional near-infrared spectroscopy (fNIRS) data from the medial frontopolar cortex (mFPC) region and the somatosensory (S1) regions of the patient and recognizing signatures in the fNIRS data associated with a pain event in real time, where the signatures include measures of dissociation between the fNIRS data from the mFPC region and the fNIRS data from the S1 region. The method further includes outputting a pain event classification to a user based on recognizing the signatures.

The foregoing and other aspects and advantages of the invention will appear in the following description. In the description, reference is made to the accompanying drawings, which form a part hereof, and in which a preferred embodiment of the invention is shown by way of illustration. Such embodiment does not necessarily represent the full scope of the invention, however, and reference is made therefore to the claims and herein for interpreting the scope of the invention.

### BRIEF DESCRIPTION OF THE DRAWINGS

The foregoing and other aspects of the present disclosure will be apparent upon consideration of the following detailed description, taken in conjunction with the accompanying drawings, which reference characters refer to like parts throughout.

FIG. 1 is a block diagram of an exemplary system for monitoring nociception, pain, and pain relief, configured in accordance with the present disclosure.

FIG. 2 is a diagram showing placement of functional near-infrared spectroscopy (fNIRS) sensors for use with the system of FIG. 1.

FIG. 3 is a side view of an exemplary fNIRS sensor layout on a patient, for use with the system of FIG. 1.

FIG. 4 is a plot illustrating graphs showing modulation of fNIRS-derived cortical responses, specifically, in the medial frontopolar cortex (mFPC) and somatosensory (S1) regions, to evoked pain stimulations by analgesics, where FIG. 4A illustrates hemodynamic responses of mFPC and S1 regions (in change in concentration of oxygenated hemoglobin HbO) over time (in seconds) to morphine following electrical pain stimulation; and FIG. 4B illustrates hemodynamics responses of mFPC and S1 (in change in concentration of HbO) over time (in seconds) to remifentanil and a placebo in anesthetized patients undergoing cardiac catheter ablation.

FIG. 5 is a graph illustrating a relationship between intraoperative brain connectivity (which is a measure of functional dissociation between mFPC and S1 in response to surgical procedures) and acute postoperative pain levels.

FIG. 6 is a plot illustrating fNIRS-derived hemodynamic responses in different pain modes, where FIG. 6A illustrates hemodynamic responses in the S1 region over time during different models (awake, sedated, anesthetized); and FIG. 6B illustrates hemodynamic responses in the left S1 region over time during different pain modalities (mechanical pain, thermal pain).

FIG. 7 is a plot illustrating the relationship between cortical hemodynamics (derived from fNIRS) and heart rate variability, HRV (derived from photoplethysmography) during pain and non-pain stimulation in an adult subjects.

FIG. 8 is a schematic view of an example display output of the system of FIG. 1.

FIG. 9 is another schematic view of an example multi-view display output of the system of FIG. 1.

FIG. 10 is a block diagram of an exemplary process flow of the system of FIG. 1.

FIG. 11 is a block diagram of an exemplary data processing pipeline of the system of FIG. 1.

FIG. 12 is a block diagram illustrating details of “Block A” of the data processing pipeline of FIG. 11.

FIG. 13 is a block diagram illustrating details of “Block B” of the data processing pipeline of FIG. 11.

FIG. 14 is a block diagram illustrating details of “Block C” of the data processing pipeline of FIG. 11.

FIG. 15 is a block diagram illustrating details of “Block D” of the data processing pipeline of FIG. 11.

FIG. 16 is a block diagram illustrating details of “Block E” of the data processing pipeline of FIG. 11.

FIG. 17 is a block diagram illustrating details of “Block F” of the data processing pipeline of FIG. 11.

FIG. 18 is a plot of simultaneous cerebral and physiological/autonomic assessments collected from a healthy volunteer during thermal pain stimulation.

FIG. 19 is a flow chart of an example method for monitoring nociception, pain, and pain relief, for example, using the system of FIG. 1.

### DETAILED DESCRIPTION

The ongoing barrage of nociception is known to induce central sensitization under anesthesia that clearly should also take into account alterations in the whole pain connectome. As such, having a quantitative method for measuring pain/nociception under anesthesia would allow for rational interventions to inhibit peripheral nociceptive drive and limit “anesthesia pain” or “unconscious pain.” That is, nociception refers the body's ability to detect a noxious stimulus, i.e., sending signals to the brain in both conscious and unconscious states in response to a noxious stimulus, whereas pain refers to a conscious person's subjective perception of the noxious stimulus in response to the brain processing the nociceptive signals. Thus, traditional methods of monitoring pain are not feasible for unconscious or sedated patients or those who have difficulty communicating, yet the body's nociceptive responses under anesthesia or sedation still correlate to post-operative acute and chronic pain. Thus, by being able to monitor nociception and mitigate nociceptive responses in unconscious, sedated, or awake patients, post-operative acute and chronic pain, as well as post-operative opioid consumption, can be better managed.

Accordingly, the present disclosure provides systems and methods for monitoring nociception and analgesia, using a combination of physiological and neurophysiological measurements including functional near-infrared spectroscopy (fNIRS), autonomic, and electrodermal activity, obtained from sensors that can be placed on a patient's scalp, forehead, and/or body. The system and method extract information from these signals related to a patient's autonomic responses to nociception, their cerebral responses to nociception, and to the patient's pharmacologic response to opioid analgesic drugs. The method uses algorithms integrating these multiple measures to produce an online, real-time evaluation to determine pain vs no pain (or decreased pain) status.

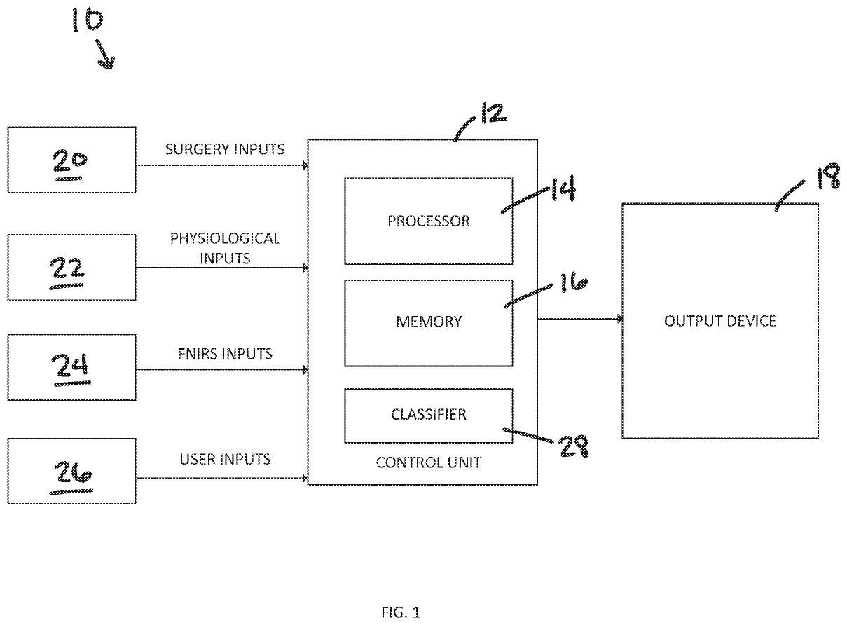

Referring now to FIG. 1, an example system 10 is illustrated, which may be configured to monitor and/or treat intraoperative nociception during a surgical procedure. Additionally, in further applications, the system 10 may be used to monitor nociception during ICU sedation (e.g., during COVID or other respiratory diseases), or to monitor inpatient post-surgical pain monitoring. Accordingly, the system 10 could be relevant to up to 240 million anesthetic cases annually worldwide, as well as an estimated 20 million ICU cases, and an estimated 120 million post-surgical patients. Furthermore, the system 10 may have further applicability for use with monitoring pain in patients who are unable to communicate (e.g., neonates, stroke patients, Alzheimer's patients, etc.).

As shown in FIG. 1, the system 10 can include a control unit 12 with a processor 14 and a memory 16 (e.g., non-transitory computer-readable memory), and an output device 18 to provide information to a user, such as a surgeon or anesthesiologist, corresponding to an objective measure of pain. In some applications, the control unit 12 can be a computing device including, but not limited to, a computer, a laptop, a tablet, etc. In other applications, the control unit 12 may be a computing device with less or different hardware than a typical computer, laptop, or tablet (but still comprising the processor 14 and the memory 16). For example, in some applications, the control unit 12 can be a small and lightweight control unit (less than 1 kilogram) that can be taped to a surgical table or placed by a patient's shoulder.

The system 10 can further include a first input device 20 to provide surgery inputs to the control unit 12, a second input device 22 to provide physiological inputs to the control unit 12, a third input device 24 to provide fNIRS inputs to the control unit 12, and a fourth input device 26 to provide user inputs to the control unit 12. Accordingly, the control unit 12 can include one or more communication channels that enables data transfer between the control unit 12 and a corresponding input device 20-26. These communication channels can be ports for wired connections, or wireless connection protocols, such as WiFi or Bluetooth.

Furthermore, generally, the memory 16 stores instructions thereon that, when executed by the processor 14, receives or acquires inputs from one or more of the input devices 20-26, via the relevant communication channel, corresponding to relevant patient data, processes the inputs for pain signal detection, applies processed inputs to a machine learning (ML) classifier 28, and outputs an objective measure of pain to a user (e.g., a surgeon) via the output device 18. Thus, when discussion herein refers to method steps or to the processor 14 taking certain actions, such discussion can further refer to the memory 16 storing instructions thereon that the processor 14 executes to take such actions. Additionally, the processor 14 can store data in the memory 16 corresponding to the various inputs, either raw or processed, and can be configured to transmit the data or the objective measure of pain to remote computing devices (e.g., for telemedicine or remote monitoring applications).

Referring still to FIG. 1, as noted above, the first input device 20 can provide surgery inputs. In one example, the first input device 20 comprises surgical video equipment, such as that associated with a surgical video display. As such, the surgical inputs can be video-based features of real-time (or substantially real-time) surgical events. In another example, the first input device 20 can be a keyboard, touch display, buttons, dials, or other input devices that enable the user to provide information corresponding to timing of surgical events or other events. Accordingly, the processor 14 can acquire the surgical inputs and identify potentially painful events or surgical manipulations based on the surgical inputs. The processor 14 can further evaluate other inputs, such as the physiological inputs and fNIRS inputs, as further described below, to calculate spatial and temporal features relating to the events identified from the surgical inputs.

As noted above, the second input device 22 can provide physiological inputs. In some applications, the second input device 22 can be a monitoring device configured to provide biopotential signals, such as, but not limited to, an echocardiogram (ECG), electromyography (EMG), electroencephalography (EEG), electro-oculogram (EOG), and/or photoplethysmography (PPG). Alternatively, or additionally, the second input device 22 can include a heart rate monitor, a respiration monitor, galvanic skin resistance monitor, or other physiological monitoring device. As such, the physiological inputs provided by data from the second input device 22 can include, but are not limited to, heart rate, respiration rate, heart rate variability, mean arterial pressure, partial pressure of oxygen, and blood pressure. As further discussed below, these physiological inputs can be correlated with fNIRS data to provide objective indications of pain during surgery.

As such, the third input device 24 can provide fNIRS inputs, e.g., cortical hemodynamics. fNIRS is a noninvasive neuroimaging technique that utilizes near infrared light (approximately 690 nanometers (nm) to 850 nm) to provide a continuous measure of cortical hemodynamics, namely, oxygenated hemoglobin (HbO) and deoxygenated hemoglobin (HbR) concentration changes. Accordingly, in some applications, the third input device 24 can include a monitoring device comprising sensors that can be placed on a patient's scalp and/or forehead to obtain fNIRS data. In some applications, the third input device 24 can include one or more probes with an array of sensors, or may include separate sensors, or one or more headbands or a cap containing the sensors.

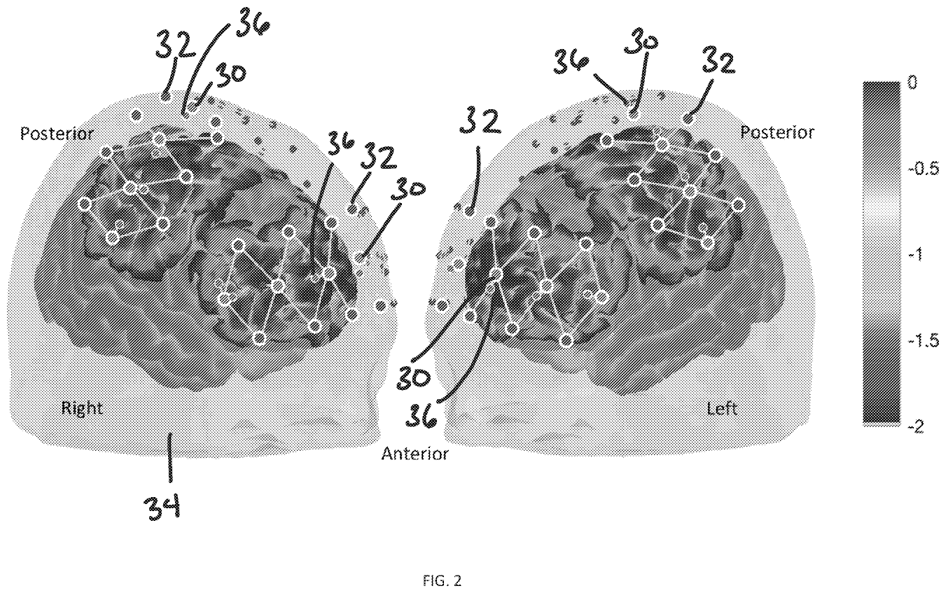

As further discussed below, simultaneous recordings of hemodynamic signals from two cortical regions, the somatosensory region (S1) and the medial frontopolar cortex region (mFPC), can be used as the fNIRS inputs to the system 10. Thus, the third input device 24 can include sensors configured to cover the patient's polar frontal and somatosensory regions. For example, FIG. 2 illustrates example placement of fNIRS emitters 30 (red) and detectors 32 (blue) on a patient 34 for surgery and corresponding cortical sensitivity. Additionally, to better characterize the physiological components in the fNIRS signals, e.g., to remove global physiological effects, short separation detectors 36 (green) can also be placed on the patient 34, which are light detectors placed at a distance of less than 1 centimeter from the light emitters 30 to capture hemodynamic signals from extracerebral layers (e.g., skin, scalp, and skull). In the illustration of FIG. 2, warmer colors indicate high sensitivity and cooler colors indicate areas of low sensitivity.

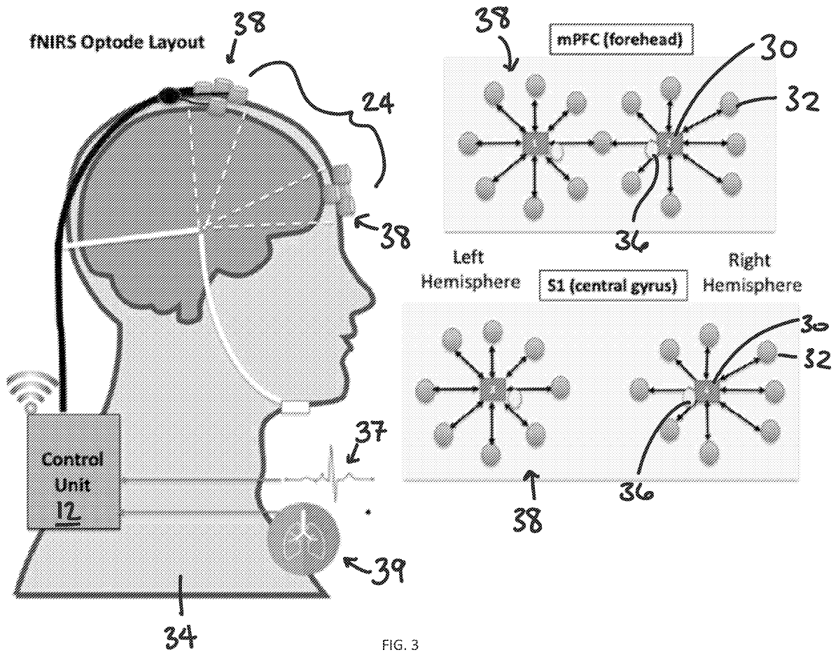

In accordance with this exemplary placement, FIG. 3 illustrates an example third input device 24 on a patient 34. In some applications, the third input device 24 can be functional with the system 10 with as little as two channel recordings from each region, i.e., four at the patient's forehead (two in each hemisphere), and four in the S1 area. In some applications, this can be accomplished via a pair of disposable probes 38 containing optodes that can be placed on the patient's scalp and forehead and secured to the patient 34. The probes can be minimal in size, such as 4 by 2 centimeters in one application. Additionally, the system 10 may be further expanded to include more channels over the mFPC and S1 regions in some applications. The optodes (i.e., the emitters 30, the detectors 32, and the short separation detectors 36) can be connected to the control unit 12, which can be taped to the surgical table or placed by the patient's shoulder. As discussed above with respect to FIG. 1, the control unit 12 can also include multiple auxiliary inputs to allow for simultaneous physiological and other stimuli inputs in synchrony with fNIRS inputs (e.g., such as EEG 37 and respiration 39, as shown in FIG. 3). Existing NIRS devices that are compatible for this application may include, but are not limited to: NIRx SPORT2, CORTECH Solutions. Accordingly, in addition to the fNIRS inputs, in some applications, the third input device 24 may also incorporate the second input device 22 by further being able to extract information related to autonomic/physiological inputs.

As noted above, the fNIRS inputs can focus on the polar frontal and somatosensory regions, as these signals, as measured by fNIRS, reflect nociception/pain. These areas are used because of the specificity of the somatosensory cortices in nociception and the potential of measuring a signal from the frontopolar areas indicating integration of nociceptive information that is not blocked with anesthetics. Previous evidence has suggested that, while there is a breakdown of brain functional connectivity during anesthesia for most systems, this is not the case for sensory connections. Furthermore, continuous or ongoing pain/nociception measurements using fNIRS-measured FPC signals can be obtained in awake volunteers as well as anesthetized patients.

Furthermore, an inverse correlation between mFPC and SI (as further discussed below) is shown to be consistent with a marker that is a) the same across different states of consciousness; b) is attenuated by opioids; and c) can be differentiated from non-painful signals (e.g., auditory) including graded responses for low vs. high pain intensities. While there may be issues of overlap in fNIRS brain responses to categories of stimuli or states (e.g., aversive), the reproducible response during awake and anesthetized states suggests these are not influenced by emotional valence, anticipation, unpleasantness, etc. Furthermore, an fNIRS mFPC signal may: (a) be specific in the context of stimulus type such as experimental or clinical persistent pain and differs in responding to these pain stimuli and to non-painful sensory (e.g., a brush stroke) or rewarding/aversive pictures (e.g., psychological stimuli); (b) evaluate both acute and persistent pain in the operating room; (c) be reversed by analgesics; (d) be consistent with existing literature (predominantly fMRI) of pain imaging using frequency analysis (specifically, low frequency fNIRS oscillations are similar to those acquired using fMRI to reflect pain states by us and others); and (e) show expected changes in central sensitization.

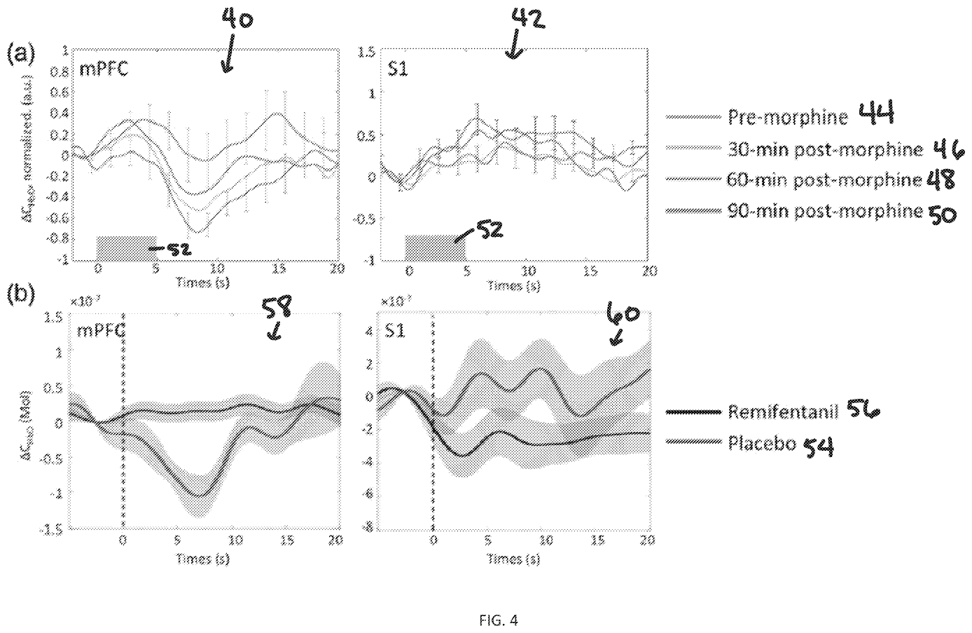

For example, FIG. 4 illustrates charts showing modulation of mFPC and S1 responses to evoked pain stimulations by analgesic measures. In FIG. 4A, data illustrates that fNIRS-derived cortical response is attenuated by opioids. In particular, the hemodynamic response of mFPC and S1 regions was attenuated by morphine but not placebo in awake healthy volunteers. That is, oral morphine reduced the amplitude of mFPC HbO decrease (chart 40) and S1 HbO increase (chart 42) associated with electrical pain stimulations in healthy, awake volunteers. In particular, line 44 illustrates baseline (pre-morphine) values; line 46 illustrates 30-minutes post-morphine administration; line 48 illustrates 60-minutes post-morphine administration; and line 50 illustrates 90-minutes post-morphine administration. The gray horizontal bar 52 indicates the timing of electrical pain stimulations. The error bars indicate the standard error of mean. The extent of amplitude reduction shown in FIG. 4A is consistent with the pharmacokinetic-pharmacodynamic model of oral morphine.

Furthermore, in FIG. 4B, data illustrates that both mFPC and S1 response was attenuated by remifentanil but not placebo in anesthetized patients undergoing cardiac catheter ablation. That is, compared with placebo (line 54), the administration of remifentanil (line 56) reduced the mFPC and S1 responses (chart 58 and 60, respectively) to invasive surgical procedures (cardiac ablation) in patients under general anesthesia.

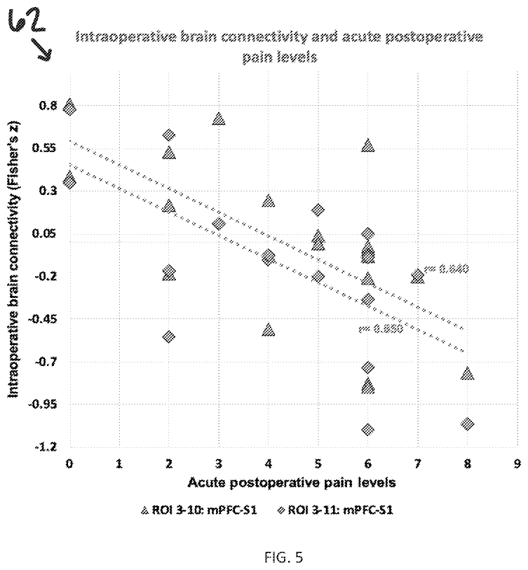

In addition to the data shown in FIGS. 4A and 4B, patients with greater dissociation between mFPC-S1 in response to procedures were shown to have greater postoperative pain. For example, FIG. 5 shows a chart 62 illustrating a negative relationship between intraoperative cortical connectivity of medial PFC and S1 regions during surgical procedures and pain levels in the post anesthesia unit. Higher negative connectivity between the two cortical regions, likely indicative of the nociceptive barrage, was associated with greater pain immediately after surgery. This could be due to suboptimal or inadequate analgesic coverage during the procedure. Accordingly, such responses, and the corresponding postoperative pain, could be mitigated by monitoring dissociation between mFPC-S1 using the present system 10. In other words, these cortical-based biomarkers could be used as inputs for the system 10, which can then output a pain or no pain indication for an anesthetist to deliver appropriate intervention during surgery, such as multimodal analgesia, and for a surgeon to understand post-surgical outcomes, e.g., such as postoperative pain levels that are potential indicators of pain chronification.

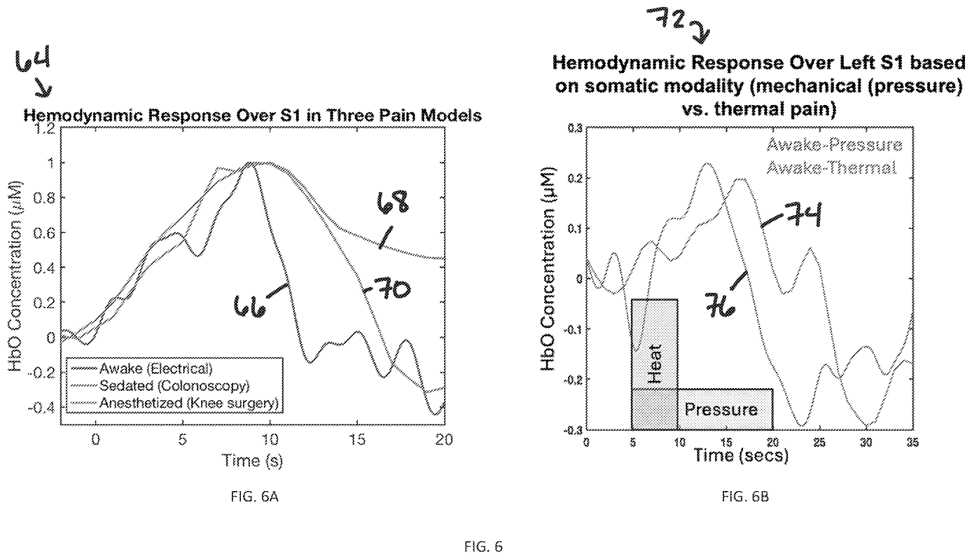

Furthermore, FIGS. 6A and 6B show additional data illustrating robust fNIRS-based measures of cortical activity during various pain modalities. More specifically, FIG. 6A illustrates a chart 64 an average hemodynamic response (i.e., change in oxyhemoglobin concentration) following various types of pain stimulation. These pain stimulation were somatic, i.e., electrical pain in awake healthy volunteers (line 66), visceral pain resulting from clinical insufflation during colonoscopy in consciously sedated patients (line 68), and visceral pain resulting from surgical tissue/bone manipulations in anesthetized patients (line 70). FIG. 6B illustrates a chart 72 comparing two different types of somatic pain stimuli to the right hand (thermal vs. mechanical stimulation) in an awake, adult subject. Five seconds of thermal pain was presented at approx. 48 deg C. (equivalent to VAS 7/10) with the resulting response shown at line 74, and pressure pain was delivered at approx. 5.2 pound-force with the resulting response shown at line 76. Both stimuli generate a similar response in the left S1 region. The faster response to pressure pain is consistent with the faster-conducting nature of type-C unmyelinated fibers that conduct dull pain such as pressure stimuli.

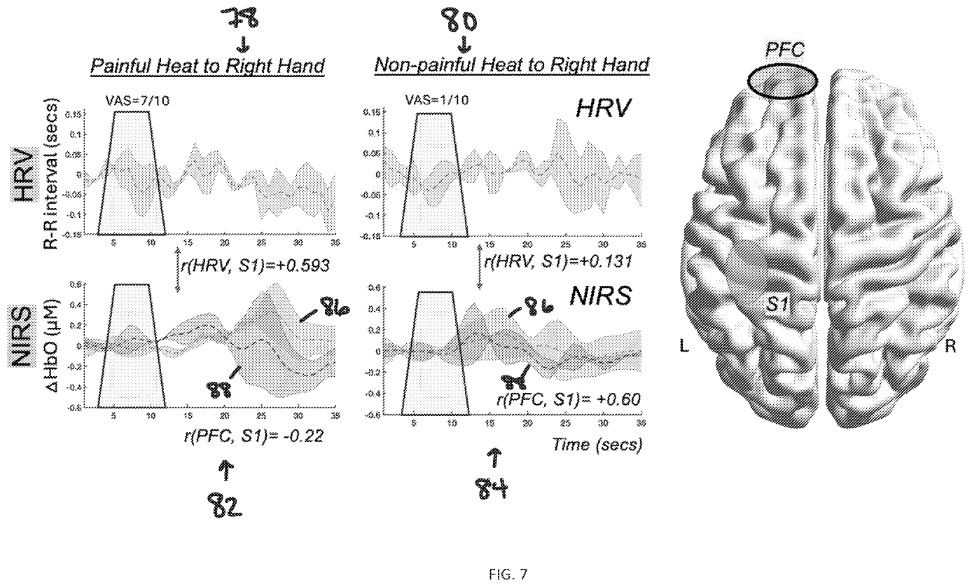

Additionally, as noted above, autonomic/physiological measures (e.g., inputs from the second input device 22) can also be correlated with cortical activity, as autonomic activation may predict pain sensation. Thus, such inputs can be used as composite measures of pain under anesthesia. For example, FIG. 7 shows data illustrating a coupling between cortical hemodynamics (derived from NIRS) and heart rate variability (HRV), derived from photoplethysmography, during pain stimulation in an adult subjects. First, average HRV following task stimulation (pain or non-painful, shown in charts 78 and 80, respectively) showing the variation in peak-to-peak interval of heart beats. The error bar represents the standard deviation across two trials of pain (VAS 7/10) or no-pain (VAS 1/10) conditions. Shaded area denotes the period of stimulation (approximately five seconds). Second, average hemodynamic changes following task stimulation (pain or no-pain, shown in charts 82 and 84, respectively) from left medial PFC (waveform 86), and left S1 (waveform 88). The NIRS signal was corrected for physiological noise by filtering and performing short-separation regression analysis. Pearson's r correlation between PFC and S1 signals showed a negative correlation (r=−0.22) during pain stimuli and a positive correlation during non-painful stimuli (r=0.60). Additionally, the S1 response and HRV changes were temporally correlated during pain (r=0.593) but not during non-pain condition (r=0.131) suggesting a coupling between the autonomic and the neurophysiological responses during pain.

Now referring back to the system 10 of FIG. 1, the fourth input device 26 can provide user inputs. Such user inputs can include, but are not limited to, analgesic information provided by a user (e.g., an anesthesiologist, a surgeon, or medical assistant) and a baseline pain threshold of the patient. For example, the fourth input device 26 can include a keyboard, touch display, buttons, dials, or other input devices that enable the user to provide analgesic information to the control unit, such as quantity and/or timing of application.

As noted above, the inputs from the input devices 20-26 are processed by the processor 14 to provide an output corresponding to an objective measure of pain via the output device 18. The details of how the processor 14 processes the inputs are discussed further below. Referring still to FIG. 1, in some applications, the output device 18 may be a display that visually illustrates this objective measure of pain to the user. In other applications, the output device 18 may be a light or audio speaker that otherwise communicates the objective measure of pain to the user.

The output can be any communication indicating an objective measure of pain. In some examples, the output can be a simple “Yes” or “No,” “Pain” or “No Pain” displayed via the output device 18. In other examples, the output can include flashing lights, colors, and/or sounds that change based on a “Pain” or “No Pain” indication. Furthermore, in some examples, rather than a binary output, the output can be illustrated as a sliding scale (e.g., bar graph, color scale, etc.), in percentiles, or with other numerical values to communicate an objective measure of pain.

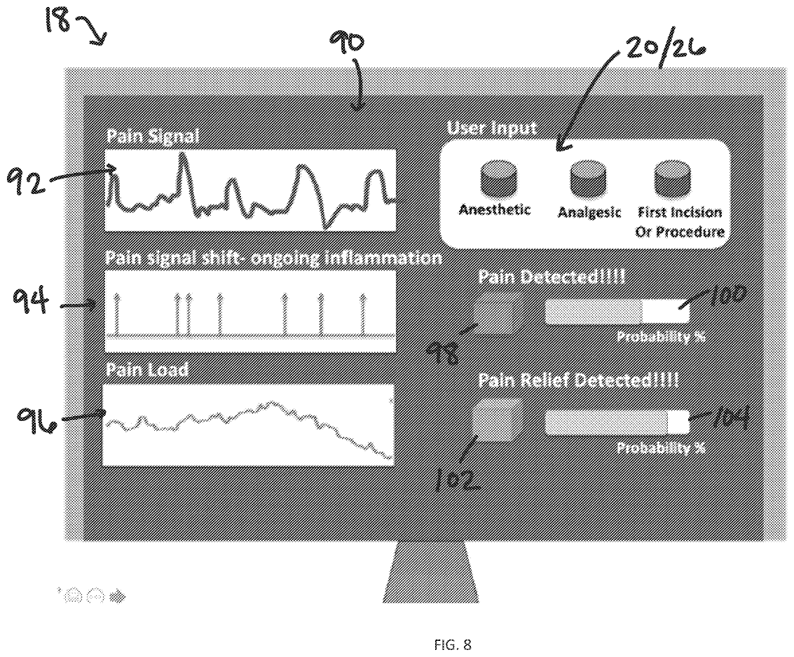

FIG. 8 illustrates an example output device 18 in the form of a monitor display. The monitor display may be part of the control unit 12 or in communication with the control unit 12 (e.g., via a wired connection or wireless connection). As shown in FIG. 8, the output can include multiple indications of pain measures, serving as a patient monitoring dashboard 90. For example, the output can include a first chart 92 illustrating a “pain signal,” a second chart 94 illustrating a “pain signal shift,” and a third chart 96 illustrating “pain load.” For example, the “pain signal” can indicate detection of pain-related signature during a surgical procedure, the “pain signal shift” can denote a shift in the cortical signature consistent with the presence of ongoing pain, and the “pain load” may be used to indicate the cumulative effect of ongoing surgical procedures. Furthermore, the output includes a first color indicator 98 (e.g., green) and a first probability scale 100 corresponding to pain detected as well as a second color indicator 102 (e.g., red) and a second probability scale 104 corresponding to pain relief detected. While the monitor display of FIG. 8 illustrates multiple outputs, it should be noted that this is but one example and more or fewer outputs can be provided in some applications.

Additionally, as shown in FIG. 8, the monitor display, while acting as the output device 18, further acts as the first input device 20 and/or the fourth input device 26. That is, the monitor display can be a touch display that receives user inputs corresponding to anesthetic or analgesic information input (i.e., corresponding to the user inputs of the fourth input device 26), and/or receives user inputs corresponding to surgical events (i.e., corresponding to the inputs of the first input device 20).

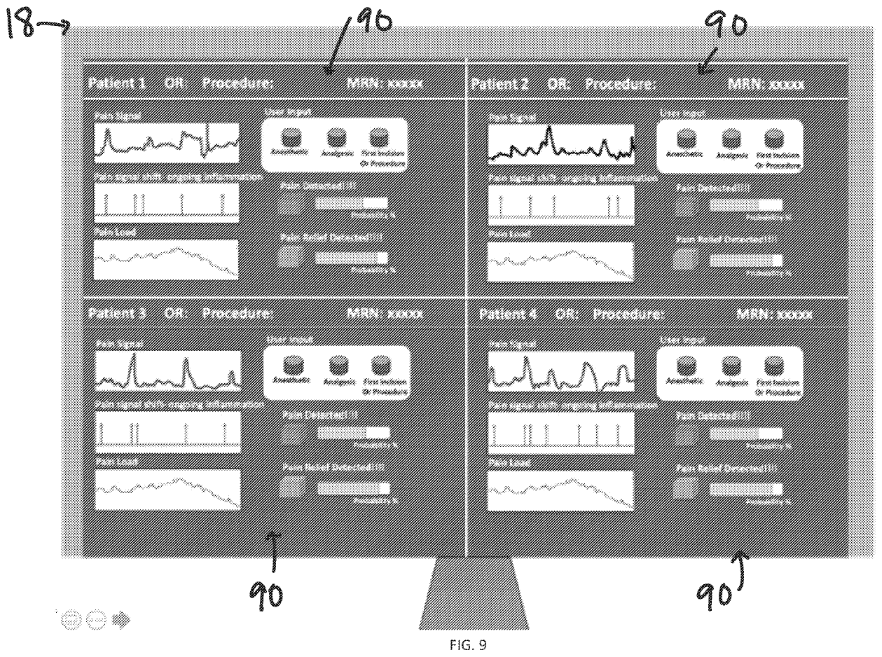

Furthermore, as shown in FIG. 9, the monitor display can serve as an output device 18 for multiple patients. That is, in FIG. 9, the control unit 12 can provide outputs via the output device 18 for four different patients via four separate dashboards 90 (or more or fewer than four in some applications). Accordingly, the system 10 may be able to provide multi-patient monitoring, for example, in a hospital environment, such as in the ICU or an OR suite.

It should be noted that, while the system 10 is described herein as comprising the control unit 12, the output device 18, the first input device 20, the second input device 22, the third input device 24, and the fourth input device 26, in some applications, the system 10 may be considered to include any combination of these components, with the other components being peripheral components that can be coupled to the system 10. Thus, in some applications, the system 10 may comprise less components than what is described herein. Furthermore, while described separately, at least one of the first input device 20, the second input device 22, the third input device 24, and the fourth input device 26 may be formed as a single device in some applications. For example, physiological sensors could be used in tandem with fNIRS (considered systemic physiology augmented fNIRS, or SPA-fNIRS) as a combination second and third input device 22/24. Additionally, one or more components of the system 10 can be remote from other components. For example, in some applications, the control unit 12 and/or the output display 18 (or a secondary output display 18 of a remote computing device) can be remote from the other components, for example, to provide objective pain measures in telemedicine or remote monitoring applications.

As noted above, the system 10 can receive the inputs from one or more of the input devices 20-26, process the inputs for pain signal detection, and output an objective measure of pain via the output device 18. That is, the system 10 uses cortical signals alone or in combination with physiological measures and surgical timing inputs to produce an online real-time evaluation to determine pain vs. no pain (or decreased pain) status. The system 10 utilizes S1 and mFPC signals, for example, in contrast to lateral prefrontal cortex signals as in previous methods. Further, in contrast to only comparing signatures of cortical signals, as in prior methods, the system 10 can utilize cortical inputs, physiological inputs, surgical inputs, and/or user inputs with an integrated machine learning classifier 28 in order to output a pain/no pain signal.

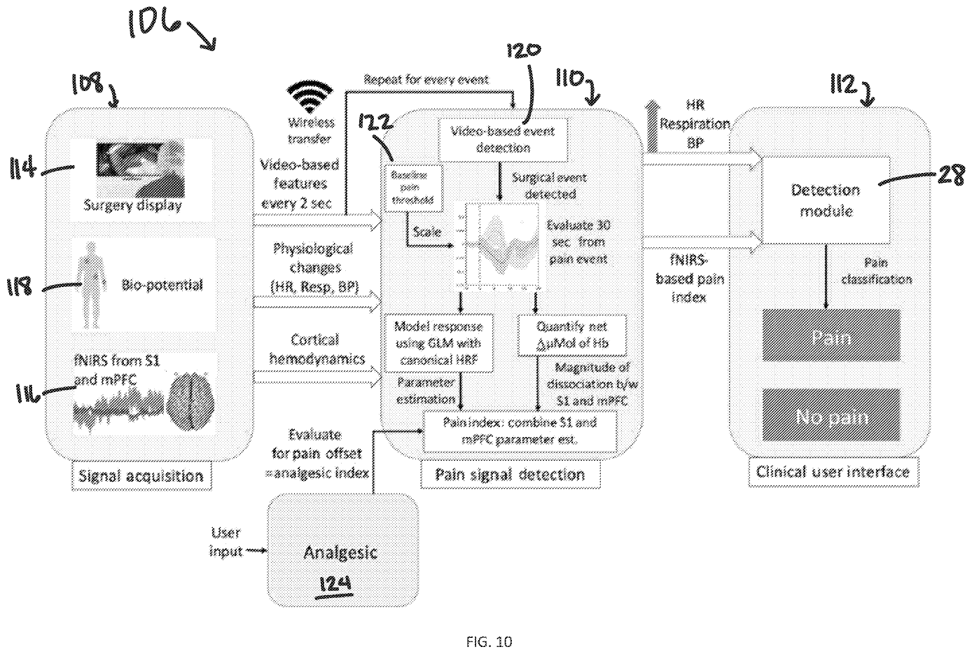

For example, FIG. 10 illustrates a process flow diagram 106 of the system 10 according to some applications. This process flow 106 provides an integrated approach to an algorithm for an objective measure of pain and is generally split into signal acquisition (block 108), pain signal detection (block 110), and user output via a user interface (block 112). Generally, during signal acquisition 108, a surgical display 114 (e.g., the first input device 20) can be connected to the control unit 12 to identify potentially painful events or surgical manipulations to feed an algorithm, which will then evaluate fNIRS data 116 (e.g., from the third input device 24) and physiological data 118 (e.g., from the second input device 22) to calculate spatial and temporal features relating to that event. These features are processed in real time, e.g., using mathematical models, for identifying signatures or features associated with pain detection (at block 110), for example based on video-based event detection data 120, an optional baseline pain threshold 122 for scaling cortical data, and user input related to analgesic application 124 for offsetting pain (e.g., inputs from the fourth input device 26). At the user interface 112, the processed data will be input into a machine learning classifier 28 to perform a binary classification corresponding to the event, i.e., pain vs. no pain.

As further detailed below, the approach illustrated in FIG. 10 allows a reasonable time delay between recorded hemodynamic signals and outputted pain measures, and therefore provides an implementation of a real-time framework for data processing. This process flow 106 can apply a sliding window method, where a data buffer is created and is used to store the recorded optical data of all available channels over a defined time-period. As new data are collected, the data buffer is updated at each time point by discarding the earliest data points and connecting the new data points to the end of the buffered time courses. Updating the buffer triggers a series of processing steps, as further described below. Furthermore, as noted above, additional inputs to improve the sensitivity of the system 10 can include a user-defined input of analgesics administered prior to or during the procedure (e.g., inputs from the fourth input device 26), a baseline pain threshold of the patient as determined prior to surgery, and use of the surgery display (e.g., the first input device 20) as input to algorithm to identify surgical events and the related biological (brain) and physiological response.

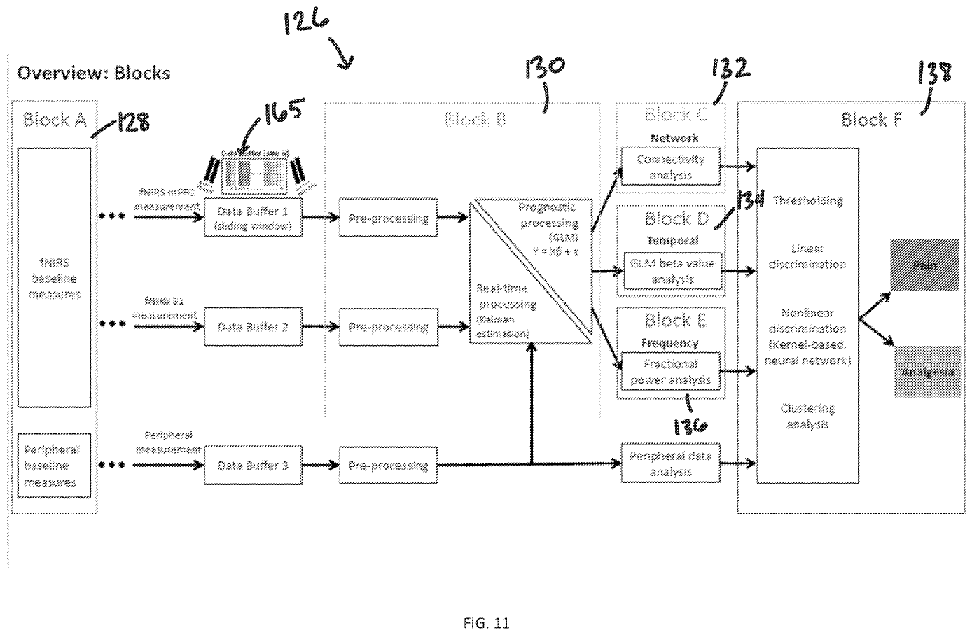

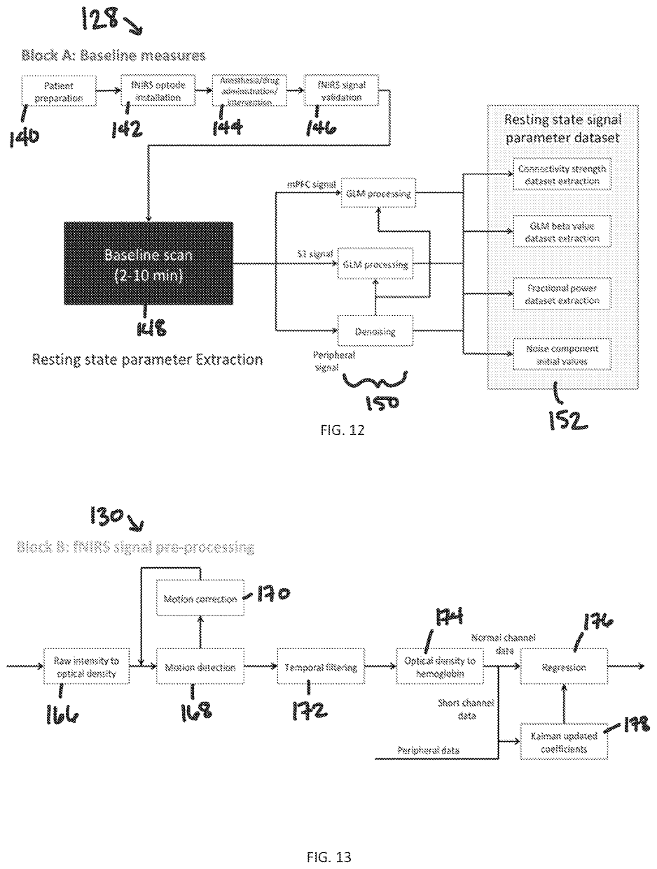

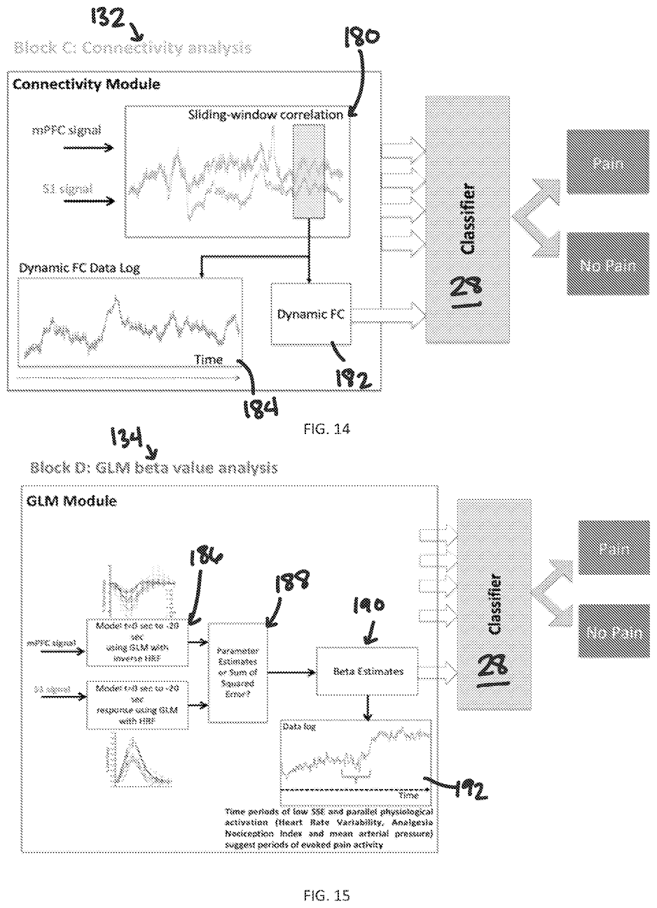

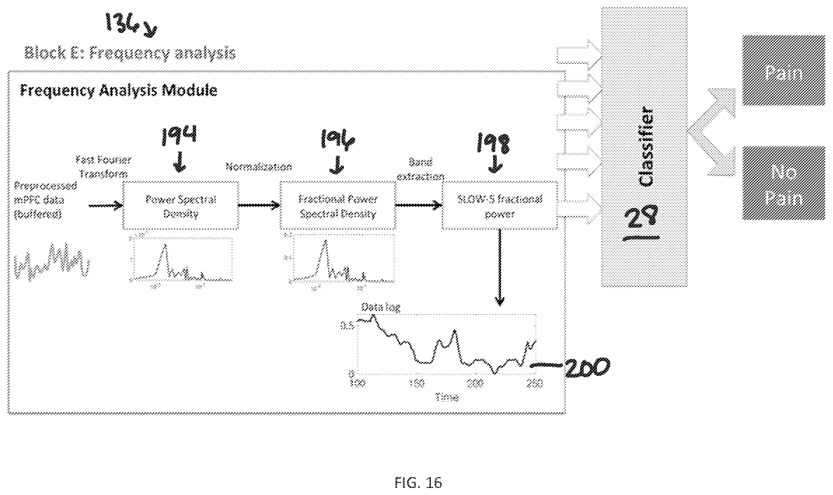

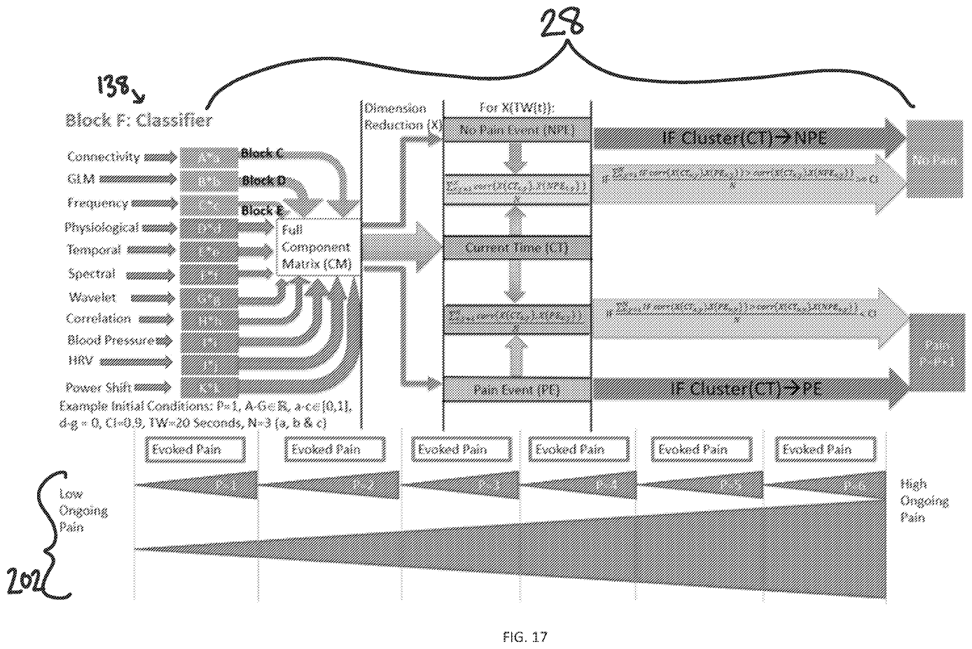

FIG. 11 illustrates an overview of a data processing pipeline 126 of the system 10, in accordance with the process flow 106 of FIG. 10. For example, according to some embodiments, the illustrated formulation of automated data collection and analysis for pain measures under anesthesia integrates a number of steps integrated into separate blocks. Generally, the data processing pipeline 126 includes a first block 128 including baseline measures (Block A, further detailed in FIG. 12), a second block 130 including fNIRS data pre-processing (Block B, further detailed in FIG. 13); a third block 132 including functional connectivity analysis (Block C, further detailed in FIG. 14); a fourth block 134 including General Linear Model (GLM)-based beta value analysis (Block D, further detailed in FIG. 15); a fifth block 136 including time-frequency analysis of low frequency oscillation fractional power (Block E, further detailed in FIG. 16); and a sixth block 138 including an integrative classifier for Pain/No Pain classification (Block F, further detailed in FIG. 17).

Referring now to FIG. 12, at the first block 128 (Block A), baseline data acquisition and analysis can be conducted to set up a dataset of resting state (i.e., baseline) signal parameters. For example, in some applications, this can be performed in a pre-surgery room. As shown in FIG. 12, Block A can include patient preparation (step 140) and fNIRS optode installation (step 142), such as installing an fNIRS cap and optodes (e.g., the third input device 24). Physiological sensors (e.g., the second input device 22) may also be installed at step 142. After the setup, anesthesiologists and nurses can conduct medical interventions such as anesthesia procedures or drug administrations to prepare the patient for the surgery (step 144).

Still referring to FIG. 12, fNIRS signal validation and adjustment can be performed (step 146). After further adjustments and validation of fNIRS measures to ensure good optode contact and signal-to-noise level, a baseline scan can be conducted for a time period (e.g., about 2-10 minutes) to obtain baseline data of an mFPC signal, an S1 signal, and a peripheral signal (step 148). A standard General Linear Model (GLM) analysis of the obtained baseline data can then be performed (step 150) to extract a dataset 152 of signal parameters in a resting state condition for the specific patient. That is, the dataset 152 forms the patient-specific baseline to compare acute or ongoing events. The dataset 512 of signal parameters can include, but is not limited to, functional connectivity strength, GLM beta values, mFPC signal fractional power (e.g., low frequency fractional power from signal frequency analysis), and noise levels. Additionally, the fNIRS data acquisition can continue as the patient is transferred from the pre-surgery room to the operation room to receive the surgery.

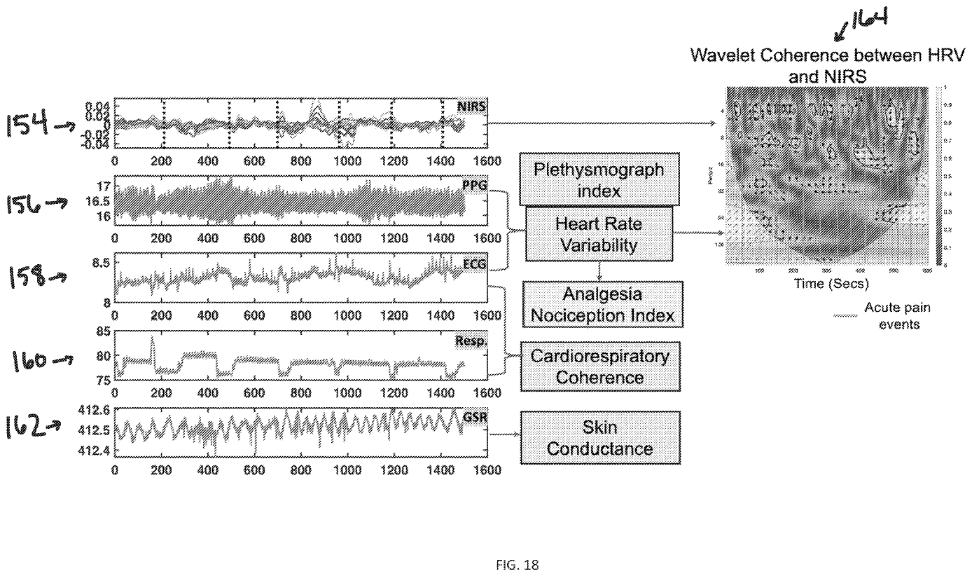

The fNIRS data, and peripheral data, can be recorded continuously until the end of the surgery. For example, FIG. 18 illustrates an example of simultaneous cerebral and physiological/autonomic assessments collected from a healthy volunteer, during thermal pain stimulation, that could be used as inputs to the system 10. The cerebral measures from NIRS (chart 154) can include cortical hemodynamic response, amplitude/power-based measures, frequency-based measures, and dynamic temporal measures. The physiological measures can be derived from blood pressure monitor, photoplethysmography (PPG) (chart 156), echocardiography (ECG) (chart 158), respiration (chart 160) and galvanic skin resistance (chart 162) and can include heart rate variability (HRV), analgesia nociception index, plethysmograph index, cardiorespiratory coherence, and skin conductance. Additionally, time-frequency coupling between both cortical and physiological signals can be obtained via wavelet coherence analysis (as shown in spectrogram 164 showing increased coherence between NIRS and HRV consistent with the task stimulation).

Referring back to the process in FIG. 11, at each time point defined by a sampling time of the fNIRS system, such as every 0.04 seconds, a data buffer 165 is updated, and the buffered fNIRS data are pre-processed at the second block 130. Additionally, as shown in FIG. 11, buffered peripheral data can be pre-processed, input to Block B, and analyzed and input to Block F (discussed further below).

Referring now to FIG. 13, at the second block 130 (Block B) where pre-processing of recorded fNIRS buffered data is conducted, the raw fNIRS optical data are first converted into optical density values (step 166). For example, raw optical intensities can be first converted into optical density changes by taking the logarithm of the time course. Motion detection and correction are then applied on the converted optical density data (steps 168 and 170, respectively) to correct sudden drifts and spikes in the timecourse coming from patient movement or optode displacement (e.g., using both signal amplitude and variance thresholds). In some applications, for artifacts that are excessive and cannot be corrected within a few iterations, the entire data in the buffer are discarded.

Referring still to FIG. 13, the motion-corrected optical density time course can undergo bandpass temporal filtering (e.g., with a typical low-pass threshold of 0.5 Hertz (Hz) and a high-pass filter of 0.01 Hz) to remove signal components that are unlikely to have a neurophysiological basis (step 172). The filtered optical density data are then transformed into hemoglobin concentration changes, e.g., using the modified Beer-Lambert Law (step 174). After calculating hemoglobin concentration changes, a short channel regression method can be applied to remove the systemic physiological noises from superficial head layers such as the scalp or the skull (step 176). More specifically, as discussed above, to better characterize the physiological components in the fNIRS signals, short separation detectors 36 can be used, which can be light detectors placed near the light emitters 30 (e.g., less than 1 centimeter away from). The short separation regression method has been shown to be effective in removing the hemodynamic signals from extracerebral layers (e.g., skin, scalp, and skull) from fNIRS-measured hemodynamic signals. For each channel, a linear model can be set up to regress extracerebral contaminations and other physiological noises using the time course of the short distance detector that has the highest correlation with the channel time course as the regressors. Furthermore, the use of Kalman filtering (step 178) by means of a temporally embedded canonical correlation analysis to perform physiological noise correction of fNIRS data can further improve online classification accuracy.

These steps at the second block 130 can be customized and modified in some applications, including modifying filtering frequency range, use of the nearest short separation channels for temporal regression, principal component analysis-based noise reduction, etc. The output of the second block 130, i.e., the pre-processed fNIRS signals, can then be further processed at the third block 132 (connectivity analysis), the fourth block 134 (GLM beta value analysis), and the fifth block 136 (fractional power analysis), and the outputs of each of those blocks can be provided to the ML classifier 28 to provide an objective pain/no pain output at the sixth block 138.

More specifically, referring now to FIG. 14, at the third block 132 (Block C), dynamic functional connectivity (FC) analysis of the preprocessed mFPC and S1 time series can be performed. In particular, the output of the third block 132 is based on a nociceptive signature that can be observed fNIRS datasets: the functional dissociation between mFPC and S1 hemodynamic response to acute pain, which is unique to noxious stimuli and diminishes following opioid administration. And such functional relationship between brain regions may be calculated using functional connectivity, defined as the statistical dependencies between signals across time. These connectivity measures, when evaluated across spatially distant anatomical regions, will allow the study of the brain as a network. Network connections, also known as connectome, can be unique markers of individual, trait, state, disease, etc. In this case, the functional connectome between the prefrontal cortex and somatosensory, and perhaps other regions, may be used as a nociception signature under general anesthesia.

Since surgery is a dynamic event where surgical events may be considered analogous to exogenous task stimuli, the third block 132 can utilize a sliding-window correlation technique 180, where the pairwise correlation of the two regions is calculated over smaller time windows. For example, using short time windows (e.g., 20 seconds) of signals incremented in 1-second durations, pair-wise correlation of brain activity from mFPC and S1 is performed to quantify the functional integration over time, also known as dynamic functional connectivity. That is, the preprocessed time series from the regions of interest (mFPC and S1) can be correlated using Pearson's r correlation within the overlapping window of twenty seconds duration to generate a log of dynamic functional connectivity 182. Additional window lengths of 10, 30, 40, 50, or 60-second durations can be used to generate secondary dynamic functional connectivity logs in parallel. The Pearson's r correlation values are then converted to Fisher-z scores using Fisher r-to-z transformation before inputting into the classifier 28. In some applications, the functional connectivity measures can also be stored in a data log 184 (in memory 16) representing the overall dynamic connectivity of these regions.

Referring now to FIG. 15, at the fourth block 134 (Block D), GLM beta value analysis of the preprocessed mFPC and S1 time series can be performed. In particular, as opposed to functional connectivity evaluation at the third block 132, the functional dissociation between mFPC and S1 may also be evaluated by dynamically comparing the activation levels of mFPC-S1 over time. For example, using a sliding-window technique, the mFPC and S1 signal over small windows can be modeled using an ordinary least square estimate with a predefined hemodynamic response as the explanatory variable. The beta estimates and model parameters between the two regions can be input to the classifier 28 to identify events of nociception-related brain response.

More specifically, a GLM 186 using a canonical hemodynamic response function (HRF) can quantify the activation level in response to acute events, although a negative HRF (denoting deactivation) can be used to model the mFPC response. The activation level (beta estimates) and other parameters of GLM at single-window level will be compared between the mFPC and S1 regions (block 188). It is hypothesized that a functional dissociation resulting from acute surgical pain will be reflected by a) negative activation in mFPC and positive activation in S1 as modeled using pre-specified HRFs, and b) low levels of sum-of-squared errors in both mFPC and S1 regions. Subject-specific HRF can be used based on a prior run or using the first surgical event as a subject baseline to improve the accuracy of this technique further. These estimates 190 will be recorded over time (to generate a GLM log 192) and inputted into the classifier 28, along with physiological responses to detect periods of evoked painful activity. In some applications, the GLM log 192 may also be stored in memory 16.

Referring now to FIG. 16, at the fifth block 136 (Block E), frequency analysis of power shifts in fNIRS signal low frequency oscillations can be performed. For example, power spectral analysis of the prefrontal cortex signal in the SLOW-5 frequency range (e.g., 0.01-0.027 Hertz (Hz)) may reveal a drop in power during periods of noxious stimulation that is not found during innocuous stimulation or resting. Accordingly, the buffered mFPC time courses can be firstly transformed into power spectral density measurements in the frequency domain with a Fast Fourier Transform (FFT) (block 194). Power spectral density (PSD) is then computed (at a normalization step) as the square of the FFT amplitude at each frequency component (block 196). The fractional power density (fPSD) of the SLOW-5 band (e.g., 0.01-0.027 Hz) is extracted by adding the PSD components of the slow-5 sub-band and normalized to the summed power of the entire frequency range (0.01-0.5 Hz) (block 198). The fPSD of SLOW-5 is then input to the classifier 28 for ongoing pain classification. In some applications, the fractional power measures can also be stored in a data log 200 in memory 16.

Referring now to FIG. 17, an example design of the classifier 28 is illustrated. That is, once each of the “n” chosen parameters have been collected and input to the classifier 28 (e.g., cortical inputs from Blocks C, D, and E, and/or peripheral physiological data), distinctions can be made within real time between a current signal that is analogous to a first pain event as opposed to a non-painful baseline period, flagging events of concern to the user. This requires a binary classification, where using a fixed number of features (which may vary from n=1 to N), the classifier algorithm classifies a given surgical period as pain or no pain state. In some applications, considering the real-time nature and potentially limited number of samples for training, the classifier 28 can be high accuracy, that is computationally efficient with low memory requirements and can continuously update with real-time data. Various classifier methods may be implemented, including, but not limited to, thresholding, linear discrimination, nonlinear discrimination (e.g., Kernel-based, neural network), clustering analysis, etc. It should also be noted that, while classifier methods such as support vector machine may be implemented, they may be computationally taxing and unsuited for real-time monitoring in the operating room.

In some applications, a feature selection may be performed using a filtering or embedded algorithm, or multiplier values or weighting factors can be assigned that determines how relevant to the categorization approach an end user wants it to be for the algorithm. For example, based on existing data, cortical-based features can be assigned greater weight than physiological data. Alternatively, one or more features can be set to 0 and ignored entirely if so desired. For example, in its simplest form, the classifier 28 may only receive inputs from Blocks C, D, and/or E, i.e., only cortical data from S1 and mFPC. This is illustrated in FIG. 17 where matrices A, B, and C are multiplied by a, b, and c, respectively, to correctly weigh the relevance of each variable to the pain reading, while the remaining variables are set to zero. As another example, as shown in FIG. 17, the classifier 28 can receive inputs from Blocks C, D, and/or E, physiological inputs, as well as spectral inputs from wavelet based time-frequency analysis that combines both cerebral and autonomic responses under anesthesia. These additional physiological inputs can be determined by analyzing peripheral data from cardiorespiratory measures including, but not limited to, heart rate, respiration rate, heart rate variability (HRV), blood pressure, partial pressure of oxygen or a combination of these measures. As another example, with reference to FIG. 17, a surgeon may want to use blood pressure, power shift, connectivity, temporal and wavelet inputs as measures to calculate when a patient is undergoing pain during surgery. The resulting matrices A, E, G, I, and K are multiplied then by a, e, g, i, and k respectively that correctly weight the relevance of each variable to the pain reading, while the remaining variables are set to zero.

The resulting vectors from the feature selection can undergo a form of dimension reduction such as Principal Component Analysis (PCA) or Linear Discriminant Analysis (LDA) to create a component matrix X in a reduced set of dimensions. From here, a linear classifier can be implemented such as logistic regression. Logistic regression uses a linear combination of the predictive features to generate the probability of a pain event. At least one to three pain events (PE) conducted initially by the surgeon is recorded for a duration equal to the chosen time window (TW, such as twenty seconds or thirty seconds). A series of TWs are also observed for a baseline region where no pain events (NPE) have been conducted. These can be the only training datasets initially input to the classifier 28 in some applications. That is, the set of pain procedures recorded (PE) and the values recorded at the baseline (NPE) form two clusters which can classify the current point of time CT. In some applications, the CT window may be associated with a detected event (e.g., via surgical video input or other user input, such as user inputs associated with analgesic applications or other inputs).

Thus, the classifier 28 can be trained with PE and NPE values and, upon receiving a CT value input, can classify CT as a pain event or no pain event. Alternatively, in another example, if 90% or more of the correlations are greater between the variables in CT and PE than between CT and NPE, this can be registered as a pain event and the pain counter can be updated (i.e., P=P+1). If the clustering or correlation conditions are not met, then no further action is taken regardless of method. Additionally, generally, the pain counter P can start at 1 upon the first pain event, with a pain multiplier p at 1. In some applications, these multipliers can be taken from a Pain Index (PI) for successive procedures that represent how the pain readouts change over successive procedures.

Additionally, in some applications, a rule-based learning classifier 28 can be implemented. For example, Pearson correlations can be taken between the variables that make up the component matrix X for CT and the series of pain and no pain TWs. If most of these correlations are shown to be more positive towards the series of pain events (PE) than the baseline events (NPE), an adverse event can be recorded. This can be defined in the equation:

$$IF \frac{\begin{matrix}{\sum }_{x , y = 1}^{N} IF corr ( X ( {CT}_{x , y} ) , X ( {PE}_{x , y} ) ) > \\ corr ( X ( {CT}_{x , y} ) , X ( {NPE}_{x , y} ) )\end{matrix}}{N} > CI \to P = P + 1$$

where the IF statement in the numerator provides a Boolean answer and the confidence interval CI is a value between 0 and 1 (0.9 and 0.95 are common examples of potential intervals). If this condition is met, the number of recorded pain events P is updated with the current time point CT recognized as a pain event. Additionally, the output of the classifier 28 (e.g., pain event detected or no pain event detected) can be displayed via the output device 18. For example, using the example output display in FIGS. 8 and 9, a “pain event” detected can be reported via the red box 102 being displayed, whereas a “no pain event” detected can be reported to the user via the green box 98 being displayed. Additionally, in some applications, the classifier 28 can output a probability of accuracy based on current data available and these probabilities can also be output to the user via the output device 18 (e.g., via the probability scales 100, 104).

In some applications, the summation of pain events, or pain counter, can be considered a “pain load” 202. A low pain load 202 (e.g., no or minimal pain events) can indicate low ongoing pain while a high pain load 202 (e.g., multiple pain events) can indicate high ongoing pain. This pain load 202 can help characterize ongoing pain after surgery as well as serve as an analgesic indicator during surgery. For example, a surgeon can monitor the output device 18 to keep the pain load 202 below a certain threshold level, which can lead to better recovery and less chronic pain after surgery. More specifically, in some applications, “pain load” may be defined as the cumulative intraoperative cerebral state or the combination of cerebral-autonomic state during surgery (or another monitoring period).

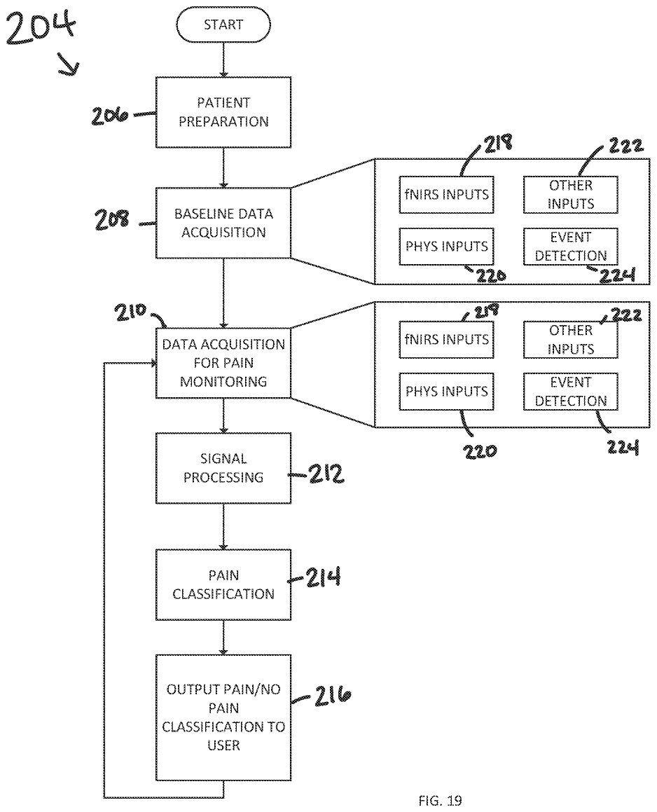

In light of the above, FIG. 19 illustrates an example method 204 of monitoring nociception, pain, and pain relief, for example, using the system 10 and processes described herein. The method 204 can be performed in various settings such as, but not limited to, during a surgical procedure to monitor nociception during the surgical procedure, following a surgical procedure to monitor ongoing post-operative pain state, during a monitoring period to monitor ongoing pain state (e.g., patient monitoring by medical professionals or patient-controlled analgesia applications), following a treatment application to evaluate a treatment (e.g., where the treatment includes one of a spinal cord stimulator, a virtual reality treatment, a nerve stimulator, a psychological treatment, and a pharmacological treatment), military applications (e.g., for analgesic administration in mass casualty environments, wounded military evacuations, etc.), space applications, submarine applications, surgical applications, clinical applications, drug development applications, drug trial applications and/or remote pain monitoring applications (e.g., telemedicine applications and/or applications for monitoring subjects in in accessible places). Generally, the method 204 includes patient preparation (step 206), baseline data acquisition (step 208), pain monitoring data acquisition (step 210), signal processing (step 212), classification (step 214), and classification output (step 216).

More specifically, at step 206, the patient is prepared, for example, by connecting the applicable input devices to the patient for acquiring cortical and other physiological inputs. This may include at least applying fNIRS probes to the patient's scalp and forehead. Additionally, signal validation can be conducted to ensure proper sensor installation. If the system 10 is being used in a surgery setting, step 206 can further include anesthesiologists and nurses conducting medical interventions such as anesthesia procedures or drug administrations to prepare the patient for the surgery.

Referring still to FIG. 19, at step 208, baseline data acquisition is conducted. This can include obtaining inputs such as at least fNIRS inputs 218 and, optionally, physiological inputs 220, other user inputs 222, and/or event detection inputs 224 that are relevant to pain detection. Additional baseline data acquisition processes that can be incorporated in step 208 are discussed above with respect to Block A and FIG. 12. Additionally, in some applications, user inputs can be acquired to associate a patient's specific baseline pain threshold with the fNIRS data to improve the sensitivity of the system 10.

At step 210, data acquisition for pain monitoring is conducted. This step can be done in a surgical setting or other settings (e.g., post-surgical, clinical, ICU, drug testing, etc.). Additionally, step 210 can include obtaining inputs such as at least fNIRS inputs 218 and, optionally, physiological inputs 220, other user inputs 222, and/or event detection inputs 224 that are relevant to pain detection, as discussed above. At step 212, the acquired data can be processed, for example, in accordance with the discussion above with respect to FIGS. 11, and 13-16 (Blocks B-D).

At step 214, associated inputs from the processed data can be input to the classifier 28 for classification, as described above with respect to FIG. 17 (Block F). The classifier 28 can be trained with relevant fNIRS data including signatures associated with pain events and baseline data, as well as other physiological data correlated with pain events and/or fNIRS data in order to classify the processed data as being associated with a pain event or no pain event. For example, in its simplest form, the classifier 28 can receive relevant fNIRS data and output a “pain” or “no pain” event by recognizing signatures in the fNIRS data associated with a pain event (e.g., dissociation between mFPC and S1). In another example, the classifier 28 can receive fNIRS data in the form of functional connectivity, GLM, and frequency analysis, and/or additional physiological data and output a “pain” or “no pain” event based on thresholding, linear discrimination, nonlinear discrimination, and/or clustering analysis.

In some applications, step 214 can be performed using data windows associated with detected events, e.g., based on video-based event detection during surgery or other user inputs, as described above. Accordingly, at step 216, the classifier 28 can output a “pain” or “no pain” classification to the user corresponding to the event. While this can be referred to as a “pain event classification,” it should be noted that the classification refers to nociception and/or pain. Additionally, in some applications, at step 216, a pain load can also be output to the user. While this can be referred to as a “pain load,” it should be noted that the load refers to nociception and/or pain. The output at step 216 can be generated at the output device 18 of the system 10. It should be noted that the output device 18 may be near the patient (e.g., in the surgical suite, ICU, or hospital monitoring stations, etc.) or remote from the patient (e.g., in telemedicine applications).

Additionally, steps 210-216 can be repeated to output pain classifications in substantially real time throughout the monitoring period or surgical procedure. For example, the method 204 can be carried out during a variety of monitoring periods such as, but not limited to, during a surgical procedure to monitor nociception during the surgical procedure, following a surgical procedure to monitor ongoing post-operative pain state, during a monitoring period to monitor ongoing pain state, e.g., for patients who cannot effectively communicate their pain levels, or following a treatment application to evaluate a treatment (such as treatment via a spinal cord stimulator, a virtual reality treatment, or a nerve stimulator, a psychological treatment, or a pharmacological treatment such as while testing the efficacy of novel drugs in clinical trials).

Additionally, in some applications, steps 210-216 can be repeated following administration of certain drugs or dosages and the corresponding outputs can be compared to assist with, for example, drug dosing evaluations, drug efficacy (e.g., vs. placebo), and head-to-head drug comparisons. Thus, in such applications, an administration step (not shown) can be performed prior to step 210 for a first administration (e.g., a first drug or a first drug dose) and the method can continue to steps 210-216 as described above. Following step 216, the method can return to the administration step for a second administration (e.g., a placebo, a second drug, or a second drug dose) and the method can continue to steps 210-216 as described above. The two outputs following the two administrations can then be compared to evaluate nociceptive responses between a first drug dose and a second drug dose; a drug and a placebo; and/or a first drug and a second drug.

Furthermore, in some applications, the method 204 can include an additional step of automatically administering analgesic measures to the patient in response to the output pain event classification and/or the pain load. For example, the system 10 can be coupled to a servo-controlled analgesic administration device such that, upon determining the pain event classification and/or the pain load, the processor 14 can control the administration of analgesics to the patient.

In light of the above, the present system, processes, and methods provide a solution to the clear need for an objective marker for pain. The system provides an objective response based on information extracted from signals related to a patient's autonomic responses to nociception, their cerebral responses to nociception, and to the patient's pharmacologic response to opioid analgesic drugs to indicate that pain is occurring during the evoked stimuli and ongoing background index of pain. This approach applies a threshold above which “pain/nociception” is determined with confidence. Objective methods such as that described herein can allow for better analysis of disease state and/or treatment efficacy. The system 10 can also set precedence for future autonomous biomarker-based pain mapping, for example, with training providing finer ranges for when pain occurs (e.g., measures of amplitude, frequency, or time-frequency or spectral measures that indicate levels of pain intensity or resolution) and how surgeons or anesthesiologists are informed in the OR by a pain monitoring system 10.

Beneficial aspects of the systems and methods described herein include, but are not limited to: use and processing of a novel fNIRS signature for opioid/analgesic drugs; use and processing of a multimodal system (brain and autonomic) for neurophysiological activity; use and processing of a novel signature for cerebral responses to nociception (dissociative relationship between mFPC (cognitive-emotional) and S1 (sensory)); integration of these features within a quantitative pain load system to predict postoperative outcomes of interest, including post-operative pain and opioid consumption; calibration of this integrated monitoring feature to minimize post-operative pain and opioid consumption; a real-time pain detection tool (providing a measure of a pain/no pain output) for use in awake, sedated, or anesthetized individuals; a “pain load” evaluation tool for awake or anesthetized/nonverbal subjects (pediatric to adult); evaluation of ongoing analgesic status; use of a thresholding method; multiple measures including physiological measures in the algorithm to enhance sensitivity and specificity; integration/modification for currently available fNIRS hardware; real-time analysis; and the use of classifiers beyond mere comparison of an acquired signal with a predetermined pattern.

While the above description is primarily directed toward monitoring pain during a surgical procedure, it should be emphasized that, by monitoring and treating intraoperative nociception, the system 10 can further minimize post-operative pain and opioid requirements. That is, the pain load output may be a predictor of post-operative outcomes of interest, including post-operative pain and opioid consumption. Furthermore, the system 10 can be useful in estimating the net surgical load that contributes toward the development in chronic post-surgical pain (CPSP) in a patient, to inform the surgeon of high-risk individuals, and to understand the cortical mechanisms relating to CPSP and its resolution following treatment (e.g., to understand drug mechanisms and treatment efficacy).

Additionally, the fNIRS technique described herein, due to its noninvasive nature, ease of setup, portability, and flexibility is an excellent tool. Further, concurrent physiological, autonomic, and cerebral hemodynamic monitoring can provide greater specificity of pain measures, as discussed above, and all of these inputs may be available from a single fNIRS system for use with methods described herein. Such techniques as those described herein have not been applied in the pain domain through time-frequency coherence analysis between the brain and physiology.

Furthermore, fNIRS can provide broad applicability to various pain modalities, such as clinical which can be visceral/somatic, and experimental which can be pain from electrical, thermal or pressure stimulation. The system 10 can measure pain objectively and, thus, having such a tool would counter bias, providing a measure when self-reports are unavailable and supplement self-reports when available. Accordingly, application of fNIRS may be considered in a domain space, such as clinical, physiological/experimental, or a drug development space.

In the clinical space, the system 10 may be used for measuring acute pain or chronic pain. Acute pain examples can include, but are not limited to trauma (tissue damage) including post-surgical pain. Chronic pain examples can include a number of conditions such as, but not limited to somatic pain such as musculoskeletal, postsurgical and posttraumatic pain, complex regional pain syndrome, neuropathic pain, cancer, chronic headaches, chronic corneal pain, visceral pain such as visceral pain syndromes (e.g., IBS, endometriosis, etc.), autonomic mediated pain (sympathetic or parasympathetic), or other pain such as genetic or metabolic (though these may be included in groups noted of above).

The ability to define an objective measure of pain in the clinic via the present system 10 would afford diagnosis, progression or regression, therapeutic efficacy, and/or treatment discontinuation/cessation. The system 10 could be used across ages, from newborn to elderly, used in patients that cannot communicate (e.g., stroke, dementia, newborn, under anesthesia including sedation), and/or used in acute pain treatment across large numbers (e.g., servosystems for acute pain medications in mass troop injuries, pain treatment in hospitals for patient-controlled analgesia, especially during sleep, etc.). Additionally, in further applications the system 10 may be extended for use in the veterinarian space.

With respect to the drug development space, the use of pain measures via the system 10 may be used to evaluate analgesics in drug development from phase 1 through phase 4, such as in the following areas: preclinical testing in animal models, first in human/phase 1 (e.g., choice of drug; target engagement; drug dosing; correlating with Pk and Pd measures; phase 2 (e.g., enhanced specificity for go-no go decisions to go onto phase 3), phase 3 (e.g., trials to enhance specificity and diminish exposure to the number of volunteers), and/or drug comparator studies.

The present invention has been described in terms of one or more preferred embodiments, and it should be appreciated that many equivalents, alternatives, variations, and modifications, aside from those expressly stated, are possible and within the scope of the invention.

As used in the claims, the phrase “at least one of A, B, and C” or “at least one of A, B, or C” means at least one of A, at least one of B, and/or at least one of C, or any one of A, B, or C or combination of A, B, or C. A, B, and C are elements of a list, and A, B, and C may be anything contained in the specification.

What is claimed is:

1. A system for monitoring acute or chronic nociception and/or pain in a patient, the system comprising:
optodes configured be placed on the patient's scalp and forehead to acquire functional near-infrared spectroscopy (fNIRS) data from the medial frontopolar cortex (mFPC) region and the somatosensory (S1) regions of the patient; and
a control unit coupled to the optodes, the control unit to:
obtain the acquired fNIRS data from the optodes;
process the acquired fNIRS data;
recognize signatures in the fNIRS data associated with a pain event in real time, wherein the signatures include measures of dissociation between the fNIRS data from the mFPC region and the fNIRS data from the S1 region; and
output a pain event classification to a user based on recognizing the signatures.

2. The system of claim 1, further comprising a physiological monitoring device configured to acquire physiological data from the patient, the physiological data including data associated with one of: echocardiogram (ECG), electromyography (EMG), electroencephalography (EEG), electro-oculogram (EOG), photoplethysmography (PPG), blood pressure, heart rate, respiration, and galvanic skin resistance,
wherein the control unit is to process the physiological data with the fNIRS data to output the pain event classification.

3. The system of claim 2, wherein the control unit is to process the physiological data with the fNIRS data by inputting the physiological data and the fNIRS data to a machine learning classifier that outputs the pain event classification.

4. The system of claim 3, wherein the control unit is to further output a pain load summation to the user based on a summation of classified pain events.

5. A system for monitoring nociception and/or pain in a patient during surgery, the system comprising:
a control unit;
a first input device to provide event detection information corresponding to a surgical event to the control unit;
a second input device to provide physiological data to the control unit;
a third input device to provide functional near-infrared spectroscopy (fNIRS) data from a medial frontopolar cortex region and a somatosensory region of the patient to the control unit; and
an output device to provide information to a user,
wherein the control unit is to:
process the event detection information, the physiological data, and the fNIRS data to obtain processed data,
input the processed data to a machine learning classifier that classifies the surgical event as a pain event or a no-pain event, and
control the output device to provide the classification from the classifier to the user.

6. The system of claim 5, wherein the first input device comprises surgical video equipment.

7. The system of claim 5, wherein the second input device measures physiological data based on at least one of echocardiogram (ECG), electromyography (EMG), electroencephalography (EEG), blood pressure monitoring, electro-oculogram (EOG), and photoplethysmography (PPG).

8. The system of claim 5, wherein the second input device is at least one of a heart rate monitor, a respiration monitor, and galvanic skin resistance monitor.

9. The system of claim 5, wherein the third input device comprises a pair of probes containing optodes, wherein the pair of probes are configured to be placed on the patient's forehead and scalp, respectively.

10. The system of claim 5, further comprising a fourth input device to provide user inputs to the control unit, wherein the user inputs correspond to at least one of: a baseline pain threshold and analgesic administration.

11. The system of claim 5, wherein the control unit is to process the fNIRS data to obtain the processed data by performing at least one of: functional connectivity analysis, General Linear Model (GLM) beta value analysis, and fractional power analysis.

12. The system of claim 5, wherein the machine learning classifier is trained using baseline data from the second input device and the third input device prior to surgery.

13. A method of detecting nociception and pain in a patient, the method comprising:
obtaining functional near-infrared spectroscopy (fNIRS) data from a medial frontopolar cortex (mFPC) region and a somatosensory (S1) region of the patient;
recognizing signatures in the fNIRS data associated with a pain event in real time, wherein the signatures include a dissociation between the fNIRS data from the mFPC region and the fNIRS data from the S1 region; and
outputting a pain event classification to a user based on recognizing the signatures.

14. The method of claim 13, wherein the pain event classification is a binary pain or no pain indication.

15. The method of claim 13, wherein recognizing the signatures in the fNIRS data includes applying a sliding-window correlation to the fNIRS data to process the fNIRS data over progressive time periods.

16. The method of claim 13, wherein recognizing signatures in the fNIRS data further comprising inputting the fNIRS data to a machine learning classifier that outputs the pain event classification.

17. The method of claim 16, further comprising obtaining physiological data from the patient and inputting the physiological data to the machine learning classifier, wherein the machine learning classifier outputs the pain event classification based on the fNIRS data and the physiological data.

18. The method of claim 17, wherein the physiological data comprises at least one of blood pressure and heart rate variability.

19. The method of claim 16, further comprising training the machine learning classifier using baseline fNIRS data from the patient corresponding to pain events and no-pain events.

20. The method of claim 13, further comprising setting a pain load value based on the pain event classification; and outputting the pain load value to the user.

21. The method of claim 13, wherein obtaining the fNIRS data from the patient includes obtaining the fNIRS data at least one of:
during a surgical procedure to monitor nociception during the surgical procedure,
following a surgical procedure to monitor ongoing post-operative pain state,
during a monitoring period to monitor ongoing pain state, and
following a treatment application to evaluate a treatment, wherein the treatment includes one of a spinal cord stimulator, a virtual reality treatment, a nerve stimulator, a psychological treatment, and a pharmacological treatment.

22. The method of claim 13, further comprising automatically administering analgesic measures to the patient in response to the pain event classification.

23. The method of claim 13, wherein obtaining the fNIRS data from the patient is performed in one of military applications, space applications, submarine applications, surgical applications, clinical applications, drug development applications, and drug trial applications.

24. The method of claim 13, wherein outputting the pain event classification to the user includes outputting the pain event classification to a remote computing device.

25. The method of claim 13, wherein:
obtaining the fNIRS data from the patient includes obtaining the fNIRS data:
after administration of a drug, and
after administration of a placebo; and further comprising:
comparing the pain event classification after administration of the drug and after administration of the placebo.

26. The method of claim 13, wherein:
obtaining the fNIRS data from the patient includes obtaining the fNIRS data:
after administration of a first dosage of a drug, and
after administration of a second dosage of a drug; and further comprising:
comparing the pain event classification after administration of the first dosage and after administration of the second dosage.

27. The method of claim 13, wherein:
obtaining the fNIRS data from the patient includes obtaining the fNIRS data:
after administration of a first drug, and
after administration of a second drug; and further comprising:
comparing the pain event classification after administration of the first drug and after administration of the second drug.
